(19) United States
(12) Patent Application Publication
(10) Pub. No.: US 2024/0325330 A1
(43) Pub. Date: Oct. 3, 2024

(54) NEW METHOD TO TREAT ACIDOSIS RELATED DISEASES WITH ACETOACETATE

(71) Applicants: INSTITUT NATIONAL DE LA SANTÉ ET DE LA RECHERCHE MÉDICALE, Paris (FR); CENTRE NATIONAL DE LA RECHERCHE SCIENTIFIQUE (CNRS), Paris (FR); UNIVERSITÉ D'ANGERS, Paris (FR); CENTRE HOSPITALIER UNIVERSITAIRE D'ANGERS, Angers (FR)

(72) Inventors: Yves DELNESTE, Angers (FR); Vincent PROCACCIO, Angers Cedex 09 (FR); Céline BEAUVILLAIN, Angers (FR); Pascale JEANNIN, Angers (FR); Pierre ASFAR, Angers - cedex 09 (FR)

(21) Appl. No.: 18/575,454

(22) PCT Filed: Jul. 7, 2022
(86) PCT No.: PCT/EP2022/069015

§ 371 Date: Dec. 29, 2023

(30) Foreign Application Priority Data
Jul. 8, 2021 (EP) 21305941.3

(51) Int. Cl. A61K 31/19

(52) U.S. Cl. CPC A61K 31/19

(57) ABSTRACT

The present invention relates to the treatment of acidosis related diseases. In this study, the inventors have investigated how human monocytes adapt, survive and differentiate into macrophages under lactic acidosis. Experiments were conducted under atmospheric oxygen and in the presence of glucose, to rule out an effect of oxygen or glucose deprivation. Prolonged exposure to LA affected monocyte/macrophage metabolism. Extracellular acidosis induced mitochondrial membrane depolarization and significantly decreased nutrient consumption, resulting in a dependence of the macrophages on a transient phase of autophagy for survival. In fasting conditions, hepatocytes produce the ketone bodies acetoacetate (AcAc) and β-hydroxybutyrate (β-OHB) that constitute alternative fuel for extrahepatic cells. The inventors found here that AcAc protected the mitochondria from acidosis-induced depolarization and mitophagy, allowing the cells to continue to metabolize nutrients, thereby avoiding the need for self-catabolism to survive. Acetoacetate therefore appears to be a crucial alternative fuel metabolite of potential therapeutic interest to increase tissue tolerance to lactic acidosis. Thus, the invention relates to the acetoacetate (AcAc) for use in the treatment of acidosis related diseases.

### FIELD OF THE INVENTION

The present invention relates to the acetoacetate (AcAc) for use in the treatment of acidosis related diseases.

### BACKGROUND OF THE INVENTION

Normal tissues have an extracellular pH (pHe) of ≈7.4. Interstitial acidification (with pHe down to 6.5) is usually observed in inflammatory processes associated to solid tumors, and infections (1-4). Indeed, rapid cell proliferation and/or hypoxia lead to an increase in glycolysis, with the release of lactic acid (LA), which dissociates, in the extracellular space, into lactate and protons, the excess of protons causing extracellular acidosis (5,6). Extracellular lactate concentrations, which range between 1.8 and 2 mM in resting tissues, can reach up to 20 mM in wounds, and 40 mM in solid tumors (6). A prolonged extracellular acidosis can affect several aspects of cellular homeostasis, including metabolism, signalling and transcriptional activities (7-9).

Macrophages are innate myeloid immune cells found in almost all tissues. They play a key role in maintaining tissue homeostasis, tissue repair, protective anti-infectious immunity, and in controlling tumor development (10). Macrophages are sometimes likened to “firefighters”, due to the essential role they play in damaged and infected areas. Through their functional plasticity, macrophages continuously adapt their phenotypes to signals (such as immune mediators and metabolites) present in their microenvironment, thereby responding precisely to the tissue needs (10-12).

In the event of severe inflammation/injury, the resident macrophages can be overwhelmed, and monocytes are therefore recruited and differentiated into macrophages (10).

The differentiation of monocytes into macrophages probably generally occurs in damaged tissues, in which LA levels may be extremely high, resulting in a low pHe. However, the differentiation of human macrophages has mostly been studied at physiological pH (pHe=7.2-7.4), which seems to be of limited relevance given that monocytes are mostly recruited to damaged sites. Human macrophages are long-lived cells, and their proliferation capacity is unclear (13,14). The ability of monocytes to survive and differentiate into macrophages under lactic acidosis conditions may therefore be crucial to ensure the maintenance of a pool of functional macrophages in damaged tissues. We and others have recently reported that LA modulates the functional phenotype of murine and human macrophages (15,16). However, the mechanisms by which monocytes/macrophages survive in prolonged lactic acidosis conditions have yet to be determined. By contrast, the metabolic adaptations by which tumor cells survive and proliferate under lactic acidosis conditions have been studied in detail (4,17-20).

### SUMMARY OF THE INVENTION

In this study, the inventors have investigated how human monocytes adapt, survive and differentiate into macrophages under lactic acidosis. Experiments were conducted under atmospheric oxygen and in the presence of glucose, to rule out an effect of oxygen or glucose deprivation. Prolonged exposure to LA affected monocyte/macrophage metabolism.

Extracellular acidosis induced mitochondrial membrane depolarization and significantly decreased nutrient consumption, resulting in a dependence of the macrophages on a transient phase of autophagy for survival. In fasting conditions, hepatocytes produce the ketone bodies acetoacetate (AcAc) and β-hydroxybutyrate (β-OHB) that constitute alternative fuel for extrahepatic cells (21). The inventors found here that AcAc protected the mitochondria from acidosis-induced depolarization and mitophagy, allowing the cells to continue to metabolize nutrients, thereby avoiding the need for self-catabolism to survive. Acetoacetate therefore appears to be a crucial alternative fuel metabolite of potential therapeutic interest to increase tissue tolerance to lactic acidosis.

Thus, the present invention relates to the acetoacetate (AcAc) for use in the treatment of acidosis related diseases. Particularly, the invention is defined by its claims.

### DETAILED DESCRIPTION OF THE INVENTION

In a first aspect, the present invention relates to the acetoacetate (AcAc) for use in the treatment of acidosis related diseases.

In a particular embodiment, the invention relates to the acetoacetate (AcAc) for use in the treatment of lactic acidosis related diseases.

As used herein, the term “acidosis related diseases” relates to diseases with acidosis that is to say diseases causing increased acidity in the blood and other body tissues. For example, acidosis related diseases are tissular hypoxias like sepsis or septic shock, intoxication to CO, hemopathies or some cancers like leukemia, lymphoma or metastatic cancer, intoxication to drug like to metformin, acute liver failure or pheochromocytoma.

Particularly, the acidosis related disease can be a lactic acidosis related disease.

Septic shock is defined by a state of shock related to an infection and which is refractory to filling and justifies the use of vaso-active amines. It is associated with an increase in lactates >2 mmol/L, characterizing lactic acidosis.

Tests for determining the capacity of disease to be a lactic acidosis related disease are well known to the person skilled in the art. In a preferred embodiment, the test consists by the biological examination carried out at the patient's bedside is the arterial blood gases allowing to measure the arterial pH and therefore to characterize the acidosis, coupled with the dosage of lactates allowing to characterize the lactic origin of the acidosis. Blood gases coupled with the lactate assay are initially performed when treating a patient in a state of shock and then repeated as necessary to monitor the effectiveness of the treatment. A correction of the pH and the concentration of lactates are favorable evolution markers.

In some embodiment the arterial blood gases and the dosage of lactate are measured.

Thus, the present invention relates to the acetoacetate (AcAc) for use in the treatment of sepsis or septic shock.

According to the invention, the term “subject” denotes a mammal, such as a rodent, a feline, a canine, and a primate. In some embodiments, the subject is a human. Particularly, the subject denotes a human with an acidosis related disease like sepsis.

As used herein, the term “sepsis” denotes a life-threatening condition that arises when the body's response to infection causes injury to its own tissues and organs. This initial stage is followed by an alteration of the immune system. Common signs and symptoms include fever, increased heart rate, increased breathing rate, and confusion. There may also be symptoms related to a specific infection, such as a cough with pneumonia, or painful urination with a kidney infection (see for example Shankar-Har et al. 2016).

As used herein, the term “septic shock”, a potentially fatal medical condition that occurs when sepsis, which is organ injury or damage in response to infection, leads to dangerously low blood pressure and abnormalities in cellular oxygenation. An official definition of the septic shock is hypotension despite optimal administration of fluids that requires administration of vasoactive drugs to maintain mean arterial pressure≥65 mm Hg and lactate>2 mmol/L (see for example Shankar-Har et al. 2016).

As used herein, the term “Acetoacetate” (AcAc) is well known in the art and relates to a 3-oxo (C4H503-) monocarboxylic acid anion which is the conjugate base of acetoacetic acid (or acetylacetic acid), resulting from the deprotonation of the carboxy group. It has a role as a human metabolite.

According to the invention, the term acetoacetate (AcAc) is used here to denote the acetoacetate and all the derivates of acetoacetate which includes but are not limited to butyl acetoacetate, ethyl acetoacetate, methyl acetoacetate, isobutyl acetoacetate, isopropyl acetoacetate, n-propyl acetoacetate, sodium acetoacetate and n-butyl acetoacetate.

In particular, the term acetoacetate (AcAc) also denotes the ethyl acetoacetate or the sodium acetoacetate.

In another embodiment, the invention relates to a method for treating an acidosis related disease in a subject in need thereof comprising administering to said subject in need thereof a therapeutically effective amount of acetoacetate (AcAc).

As used herein, the term “treatment” or “treat” refer to both prophylactic or preventive treatment as well as curative or disease modifying treatment, including treatment of subjects at risk of contracting the disease or suspected to have contracted the disease as well as subjects who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at a regular interval, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

### Therapeutic Composition

Another object of the invention relates to a therapeutic composition comprising the acetoacetate (AcAc) for use in the treatment of acidosis related diseases in a subject in need thereof.

Any therapeutic agent of the invention may be combined with pharmaceutically acceptable excipients, and optionally sustained-release matrices, such as biodegradable polymers, to form therapeutic compositions.

“Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

The form of the pharmaceutical compositions, the route of administration, the dosage and the regimen naturally depend upon the condition to be treated, the severity of the illness, the age, weight, and sex of the patient, etc.

The pharmaceutical compositions of the invention can be formulated for a topical, oral, intranasal, parenteral, intraocular, intravenous, intramuscular or subcutaneous administration and the like.

Preferably, the pharmaceutical compositions contain vehicles that are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The doses used for the administration can be adapted as a function of various parameters, and in particular as a function of the mode of administration used, of the relevant pathology, or alternatively of the desired duration of treatment.

In addition, other pharmaceutically acceptable forms include, e.g. tablets or other solids for oral administration; time release capsules; and any other form currently can be used.

Pharmaceutical compositions of the present invention may comprise a further therapeutic active agent. The present invention also relates to a kit comprising an agonist, antagonist, or inhibitor of the expression according to the invention and a further therapeutic active agent.

For example, an anti-acidosis related diseases agents can be added to the present therapeutic composition. An anti-acidosis related diseases agents can be for example anti-sepsis agents or anti-sepsis shock agents.

As used herein, anti-sepsis agents or anti-sepsis shock agents can be for example antibiotics or vasopressors (like norepinephrine or dopamine).

The invention will be further illustrated by the following figures and examples.

However, these examples and figures should not be interpreted in any way as limiting the scope of the present invention.

### FIGURES

FIG. 1. Acetoacetate prevents the decrease of intracellular ATP induced by acidosis. A. Monocytes were polarized into macrophages in the absence (Mφ) or presence of lactic acid (LA-Mφ), or both lactic acid and acetoacetate (LA-Mφ+AcAc). Macrophages with or without depolarized mitochondria (“Depol” and “Pol” populations, respectively) were sorted from day 3 LA-Mφ by flow cytometry using MitoTracker Green and MitoTracker Deep Red probes. Purity was >99%. Intracellular ATP levels were measured using a semi-quantitative assay kit. Results are normalized on the Mφ condition for each donor (Mean±SEM, n=5). *p<0.05; **p<0.01 (Mann-Whitney test).).

B. PBMC from patients with septic shock were cultured for 24 h in medium containing 0.5 g/L glucose supplemented or not with 5 mM of acetoacetate (AcAc) or with 2 g/L of glucose (Glc). Intracellular ATP levels were measured using a semi-quantitative assay kit. Results are normalized on the 0.5 g/L Glc condition for each patient (n=7).

A,B. Statistical significance was determined using one-way ANOVA followed by holm-sidak's multiple comparisons test (A) or 2-tailed Student's t test with Welch's correction (B). Data are presented as mean±SEM. * P<0.05; ** P<0.01; ns, not significant.

FIG. 2. Acetoacetate prevents acidosis-induced pseudostarvation. A-E. Monocytes were polarized into macrophages in the absence (Mφ) or presence of lactic acid (LA-Mφ), both lactic acid and acetoacetate (LA-Mφ+AcAc) or under acidosis (HCl-Mφ). Glucose (A), lactate (B), glutamine (C), and amino acids (D) were quantified on days 3, 5 and 7 in cell culture supernatants of Mφ, LA-Mφ, LA-Mφ+AcAc and HCl-Mφ. Results are expressed in mmol/L/24 h, with positive values for consumption and negative values for production (n=5). E. Intracellular pH was measured by flow cytometry with the SNARF probe. Monocytes were loaded with the SNARF probe and analyzed for 30 seconds before the addition (arrow) of 10 mM lactic acid (LA-Mφ) with or without 5 mM acetoacetate (LA-Mφ+AcAc). Acquisition was then prolonged for an additional 30 minutes. Probe loading and acquisition were repeated at 24 h. Representative results from one of three independent experiments are shown. F. Acetoacetate (AcAc) was quantified on days 1 and 3 in LA-Mφ+AcAc culture supernatants (n=5). A-F. Mean±SEM. *p<0.05; **p<0.01; ***p<0.001 (Mann-Whitney test).

FIG. 3. Impact of acetoacetate on the phenotype of LA-Mφ. Monocytes were differentiated into macrophages in the absence (Mφ) or presence of lactic acid (LA-Mφ), with or without acetoacetate (AcAc). At day 5, cells were stimulated for 16 h with LPS and cytokines were quantified by ELISA in the supernatants (n=5). Mean±SEM. *p<0.05; **p<0.01; ***p<0.001 (Mann-Whitney test).

FIG. 4. Acetoacetate prevents mitochondrial membrane potential (ΔΨm) depolarization and apoptosis in septic shock. A-F. ΔΨm depolarization and apoptosis of freshly purified (A,D) or cultured (B,C,E,F) PBMC from healthy subjects or sepsis patients were analyzed by flow cytometry. PBMC were cultured for 24 h in medium containing 0.5 g/L glucose and supplemented with 5 mM of acetoacetate (AcAc) or with 2 g/L of glucose (Glc). Mean±SEM. *p<0.05; **p<0.01 (Mann-Whitney test).

Statistical significance was determined using 2-tailed Student's t test with Welch's correction. Data are presented as mean±SEM. * P<0.05; ** P<0.01; *** P<0.001; ns, not significant.

### EXAMPLE

### Material & Methods

### Monocyte Isolation and Macrophage Generation

Peripheral blood mononuclear cells (PBMC) were obtained from healthy human volunteers (Blood collection center, Angers, France; agreement ANG-2017-01) by standard density-gradient centrifugation on lymphocyte separation medium (Eurobio, Courtaboeuf, France). CD14+ monocytes were then isolated by positive magnetic cell-sorting (Miltenyi Biotec, Bergisch Gladbach, Germany). Monocytes (1×10⁶ cells/mL) were cultured in complete medium (CM) consisting of RPMI 1640 (Lonza, Verviers, Belgium) supplemented with 10% fetal calf serum (FCS) (Eurobio), 2 mM L-glutamine, 1 mM sodium pyruvate, 0.1 mM non-essential amino acids, 10 mM HEPES, 100 U/mL penicillin and 100 μg/mL streptomycin (all from Lonza) in the presence of 50 ng/mL GM-CSF (R&D Systems, Minneapolis, MN).

Experiments were performed in the presence of 10 mM lactic acid (Sigma-Aldrich, St Louis, MO) resulting in a pHe of 6.5 to generate LA-Mφ (lactic acidosis conditioning), 12.35 mM HCl to reach a final pHe of 6.5 and to generate HCl-Mφ(acidosis conditioning), 10 mM sodium lactate (Sigma-Aldrich) to generate lactate-Mφ (lactosis conditioning, pH 7.3), in the absence or presence of 5 mM lithium acetoacetate (AcAc) (Sigma-Aldrich, St Louis, MO).

### Measurement of Mitochondrial Membrane Potential

Mitochondrial membrane potential was assessed by incubating with 10 nM MitoTracker Green and 5 nM MitoTracker DeepRed (Thermo Fisher Scientific, Waltham, MA) in PBS containing 1% (w/v) BSA in PBS (PBS/BSA) at 37° C. for 15 min. Cells were washed and incubated with 0.5 μg/mL DAPI to exclude dead cells. Flow cytometry data were acquired with a FACSCanto II flow cytometer (BD Biosciences, San Jose, CA) and analyzed with FlowJo software (Tree Star, Ashland, OR).

### Measurement of Intracellular pH

Intracellular pH (pHi) was measured by incubating 3×10⁵ Mφ with 500 nM pH-sensitive dye carboxy-SNARF-AM (Life Technologies, Carlsbad, CA) in PBS/BSA at 37° C. for 20 min and then performing flow cytometry analysis. We determined pHi as the ratio of fluorescence intensities at two emission wavelengths (585/42 nm and 700/60 nm PMTs). SNARF fluorescence in macrophages was calibrated with a high-potassium buffer (39.6 mM NaCl, 120 mM KCl, 2.3 mM CaCl₂), 1 mM MgCl₂, 5 mM HEPES, 10 mM glucose) at pH values of 6.5 to 8, in the presence of 10 μM nigericin (Sigma-Aldrich); nigericin exchanges external potassium with internal protons to equilibrate extracellular and intracellular pH.

Analysis of mRNA Levels

Cells were lysed in Trizol reagent (Life Technologies, Thermo Fisher Scientific), and total RNA was extracted with the RNeasy Micro kit (Qiagen, Hilden, Germany) and then reverse-transcribed with the Superscript II reverse transcriptase (Life Technologies). Levels of mRNA encoding the indicated proteins were then analyzed by subjecting the cDNA obtained by reverse transcription to qPCR. Relative quantification was performed by the 2^{-ΔΔCT} method, with RPS18, EF1A, TBP, RPL13A and PPIA as references. Results are expressed as relative mRNA levels.

### Oxygen Consumption Rate (OCR)

We used an XF96 extracellular flux analyzer (Agilent Technologies, Santa Clara, CA) to determine the bioenergetic profile of cells. Day-4 cells were used to seed XF96 plates (50×10³ cells cells/well) and were allowed to recover for 24 h. Cells were then incubated in bicarbonate-free DMEM (Sigma-Aldrich) supplemented with 11 mM glucose, 2 mM L-glutamine and 1 mM sodium pyruvate, in a CO₂-free incubator for 1 h. Oxygen consumption rate (OCR) was recorded, to assess mitochondrial respiratory activity and glycolytic activity. OCR was recorded in basal conditions, and the cells were then treated sequentially with 2 μg/mL oligomycin, and 3 μM carbonyl cyanide p-(trifluoromethoxy) phenylhydrazone (FCCP) (both from Sigma-Aldrich). Non-mitochondrial respiration (OCR after treatment with 1 μg/mL antimycin A (Sigma-Aldrich) was subtracted from all OCR measurements. ATP-linked respiration was estimated from the difference between the basal and oligomycin-inhibited respiration rates, and proton-leak respiration was obtained by subtracting non-mitochondrial respiration from the OCR measured after oligomycin treatment. Maximal respiratory capacity was determined as the rate of respiration in the presence of the mitochondrial oxidative phosphorylation uncoupler FCCP. Three independent replicates of each measurement were generated, and results were normalized according to cell concentration.

### Western Blot Analysis

Levels of LC3-I, LC3-II, p62, β-actin, AMPKα and pAMPKα were evaluated by western blotting. Cells were lysed in RIPA buffer containing protease inhibitors (Roche Applied Science, Penzberg, Germany). When indicated, cells were treated with 10 mM BafA1, 6 h before lysis. Lysates were centrifuged at 12 000×g for 10 min at 4° C. to remove cell debris. Proteins (10 μg/lane) were separated by electrophoresis in a 4-20% polyacrylamide gel (Bio-Rad, Hercules, CA) in reducing conditions before transfer to a nitrocellulose membrane (Bio-Rad). Membranes were saturated in TBS/5% BSA/0.1% Tween 20, and then incubated for 16 h at 4° C. with polyclonal rabbit anti-LC3 or anti-phospho-AMPKα (Thr172) antibodies (Cell Signaling Technology, Danvers, MA) or with a mouse anti-p62 Ick ligand antibody (BD Biosciences, San Jose, CA). Protein loading was assessed by probing the membrane with a rabbit anti-β-actin antibody (Abcam, Cambridge, UK). Bound antibodies were detected by incubation with 1 μg/mL peroxidase-conjugated anti-rabbit or anti-mouse IgG antibody (Life Technologies) or with an anti-AMPK Ab, with the SuperSignal West Femto system (Thermo Fisher Scientific). Genetools software (version 4.01) from Syngene was used to quantify band intensity.

### Mitochondrial Enzyme Activities

The activities of complex IV of the respiratory chain and of citrate synthase were measured as described elsewhere (22). Briefly, cell pellets (3×10⁶ cells) were resuspended in cell buffer (250 mM sucrose, 20 mM Tris, 2 mM EDTA, 1 mg/mL BSA, pH 7.4). They were subjected to a freeze-thaw cycle, and then centrifuged (15000×g, 1 min). The pellet was resuspended in the same volume of cell buffer. For complex IV activity (cytochrome c oxidase), GM-Mφ and GM+LA-Mφ were resuspended in cell buffer at densities of 1×10⁵ cells/mL and 2×10⁵ cells/mL, respectively, and 0.05 mM reduced cytochrome C, 1 mg/mL BSA and 0.25 mM laurylmaltoside were added to the reaction mixture. The rate of reduced cytochrome c oxidation was monitored at λ=550 nm. Citrate synthase activity was measured as follows: the prewarmed reaction mixture (0.15 mM DTNB, 0.5 mM oxaloacetic acid, 0.3 mM acetyl coA, 0.1% (v:v) Triton X100) was added to 2×10⁵ Mφ and the rate of appearance of CoA-SH was measured at λ=412 nm.

### Metabolite Quantification

The levels of glucose, glutamine, free L-amino acids and lactate in cell culture supernatants, and of acetyl-CoA in whole-cell lysates were determined, together with the intracellular ADP/ATP ratio, with enzymatic assays (Abcam), according to the manufacturer's instructions. Results are expressed in mmol/L/24 h, with positive values indicating consumption and negative values indicating production. Acetoacetate concentrations were quantified with β-hydroxybutyrate dehydrogenase (3-HBDH) (Roche Applied Science), an enzyme that catalyzes the reversible reduction of acetoacetate to β-hydroxybutyrate in the presence of excess NADH, with the concomitant oxidation of NADH. The progress of the reaction was monitored by measuring the decrease in 340 nm absorbance due to the conversion of NADH into NAD. AcAc consumption was determined by subtracting the concentration of AcAc in cell culture supernatants from that in cell-free culture supernatants.

The intracellular levels of ATP in whole-cell lysates were determined with enzymatic assays (Abcam, Cambridge, UK), according to the manufacturer's instructions. ATP was quantified in (i) macrophages generated in the absence (Mφ) or presence of lactic acid (LA-Mφ) or of lactic acid and acetoacetate (LA-Mφ+AcAc), (ii) macrophages with or without depolarized mitochondria (“Depol” and “Pol” populations, respectively) isolated from day 3 LA-Mφ by flow cytometry using MitoTracker Green and MitoTracker Deep Red probes (purity>99%) and, (iii) PBMC isolated from septic shock patients (n=3) were cultured for 24 h in a medium containing 0.5 g/L glucose, supplemented with 5 mM of acetoacetate (AcAc) or with 2 g/L of glucose (Glc). Results are normalized on the condition 0.5 g/L glucose (None).

Quantification of mtDNA Copy Number

Mitochondrial DNA (mtDNA) content was determined by qPCR with primers specific for the ND4 and COX1 genes, and was weighted according to nuclear DNA levels, which were quantified by analyzing P2-microglobulin, as previously described (23).

### Macrophage Stimulation and Cytokine Quantification

Day 5 GM-Mϕ, GM+LA-Mϕ and GM+LA-Mϕ+AcAc were stimulated with 200 ng/mL LPS (Sigma-Aldrich). Cytokines were quantified by ELISA (R&D Systems) in the supernatants collected at 16 h.

### Determination of Relative Cell Size

Cell diameter was determined with the cell size analysis function of the Cellometer Auto T4 cell counter (Nexcelom Biosciences, Lawrence, MA); for each sample (n=3), 150 cells were measured. Relative cell size was determined by flow cytometry with the FSC-A parameter and the exclusion of non-viable cells by 7-AAD staining. About 5000 cells were measured (n=7).

### Confocal Microscopy and Image Analysis

Cells were first incubated with 150 nM MitoTracker Green (Thermo Fisher Scientific) in CM medium at 37° C. for 10 min. The mitochondrial network was characterized as described by Iannetti et al (70). Briefly, images were acquired on a Leica SP8 confocal microscope (Leica Microsystems, Nanterre, France) and submitted to a routine script in Matlab R2014a (The Mathworks, Natick, CA) for top-hat filtering (7×7 kernel), followed by median filtering (3×3 kernel) and binarization with the Otsu algorithm. Image blobs were then analyzed with the regionprops function of Matlab.

### Electron Microscopy

We fixed 5×10⁶ macrophages by incubation for 16 h at 4° C. with 2.5% electron-grade glutaraldehyde (LFD Distribution, Sainte Consorce, France) in 0.1 M phosphate buffer pH 7.4. Samples were rinsed with 0.1 M phosphate buffer and post-fixed by incubation with 1% osmium tetroxide for 45 min at room temperature. Samples were then dehydrated in graded series of ethanol solutions and finally embedded in Epon at 60° C. for 48 h. Embedded samples were cut into 60 nm-thick sections, which were contrast-stained with 3% uranyl acetate in water for 10 min and then observed under a Jeol JEM 1400 transmission electron microscope operating at 120 keV and equipped with a Gatan Orius digital camera. We obtained full cross-sections of macrophages at high resolution by acquiring multiple fields at a magnification of 20,000× and stitching them together in Adobe Photoshop with the Photomerge routine. Stitched images were reviewed and scored by a trained electron histopathologist for the presence of autophagy and mitophagy vacuoles. Mitochondrial size was determined by measuring the maximum Feret diameter on TEM images.

### Cell Apoptosis

Apoptosis was assessed by flow cytometry in freshly purified PBMC or after 3-day culture with or without AcAc. Cells were labeled with 7-AAD, Annexin-V FITC and anti-CD14 APC monoclonal antibody (all from BD Biosciences, San Jose, CA) and apoptotic monocytes were defined as CD14+ Annexin-V+ cells.

### Patient Samples.

Peripheral blood mononuclear cells (PBMC) from septic patients and healthy subjects were purified by standard density-gradient centrifugation on lymphocyte separation medium (Eurobio, Courtaboeuf, France). PBMC were cultured for 24 h in a medium consisting of RPMI 1640 medium with L-glutamine and without glucose (Lonza, Verviers, Belgium) and supplemented with 6% FCS (Eurobio), 3% autologous plasma, 0.5 g/L glucose, 100 U/mL penicillin and 100 μg/mL streptomycin (all from Lonza) in the presence of 2 ng/mL GM-CSF (R&D Systems). In some experiments, the culture medium was supplemented with 5 mM AcAc or adjusted to 4 g/L glucose. After 24 h culture, the percentage of cells with depolarized mitochondria was assessed as described above. After 3 days of culture, intracellular ATP has been quantified as described above.

### Statistical Analysis

The results are presented with histograms (mean±SEM) or with Tukey's box and whisker plot. Statistical analyses were performed with PRISM version 7.02 (www.graphpad.com). The non-parametric Mann-Whitney test was used to compare two conditions. p values<0.05 were considered statistically significant and n refers to different individual donors.

### Results

### Human Macrophages Display a Reduced Mitochondrial Mass in Lactic Acidosis.

We investigated the mechanisms by which human monocytes cope with LA by culturing monocytes with GM-CSF (Mϕ) without or with 10 mM LA (LA-Mϕ; pH=6.5), the maximum non-toxic concentration for Mϕ (data not shown). This concentration of LA and a pH of 6.5 are commonly observed in inflamed tissues (1,6).

We first compared the mitochondrial respiration of macrophages, by measuring the oxygen consumption rate (OCR) of day 4 Mϕ and LA-Mϕ. Basal respiration, ATP-linked respiration and maximum respiratory capacity were significantly lower in LA-Mϕ than in Mϕ (data not shown). However, the ATP-linked respiration/maximal respiration and proton leak/maximal respiration ratios were similar in LA-Mϕ and Mϕ (data not shown). These results suggest that mitochondrial mass was diminished in day 4 LA-Mϕ. Consistent with this hypothesis, mitochondrial DNA (mtDNA) copy number, an indicator of the mitochondrial mass, decreased markedly from day 3 to day 5 in LA-Mϕ, subsequently displaying a trend towards normalization at day 7 (data not shown). Accordingly, the activity of citrate synthase, another marker of mitochondrial mass, was also reduced in day 4 LA-Mϕ compared to Mϕ (data not shown). By contrast, the residual mitochondria maintain a quite functional electron transport chain (ETC), as the part of the maximal capacity used for sustaining ATP synthesis is not significantly decreased and the complex IV activity normalized to citrate synthase activity was not affected (data not shown).

Lactic acidosis results from the extracellular accumulation of lactate and protons. We therefore investigated whether the decrease in mitochondrial mass observed in LA-Mϕ (10 mM LA, pH=6.5) was due to acidosis or lactosis. We determined the mtDNA content in macrophages generated under conditions of acidosis (HCl-Mϕ; 12.3 mM HCl, pH=6.5) or lactosis (lactate-Mϕ; 10 mM sodium lactate, pH=7.3) alone. We found that the mtDNA copy number was significantly reduced in macrophages under acidosis, but not under lactosis (data not shown). In subsequent experiments, to mimic the pathophysiological situations, we have generated macrophages under lactic acidosis.

### The Expression of Genes Involved in Mitochondrial Biogenesis is Unaffected by Lactic Acidosis.

Mitochondria undergo constant remodeling to maintain correct function. A decrease in mitochondrial mass may therefore result from a reduction of mitochondrial biogenesis and/or an increase in mitophagy (clearance of dysfunctional mitochondria by the autophagic machinery) (25,26). We investigated the process affected by LA, by analyzing the impact of LA on the expression of genes involved in mitochondrial biogenesis. The expression of TFAM, NRF2 and PGC-1β did not significantly differ between LA-Mϕ and Mϕ, whatever the time-point analyzed; PGC-1α was poorly expressed (data not shown). These results suggest that the decrease in mitochondrial mass in LA-Mϕ does not result from a reduction in mitochondrial biogenesis.

### Lactic Acidosis Induces Mitochondrial Dysfunction.

Mitochondrial depolarization to a dysfunctional level below a certain membrane potential is a prerequisite for mitophagy (27). We therefore evaluated the presence of dysfunctional mitochondria displaying a loss of mitochondrial membrane potential (ΔΨm) in Mϕ and LA-Mϕ. We distinguished between respiring mitochondria and dysfunctional mitochondria, through a combination of staining with MitoTracker Green (total ΔΨm-independent mitochondrial content dye) and MitoTracker Deep Red (ΔΨm-dependent mitochondrial dye, suitable for use in an acidic medium) (28-30). We observed a massive accumulation of depolarized mitochondria (MitoTracker Greenhigh MitoTracker Deep Redlow) in LA-Mϕ, peaking on day 3 and in HCl-Mϕ but not in lactate-Mϕ (data not shown).

Fission events, which segregate dysfunctional mitochondria and generate smaller mitochondria, are a prerequisite for the elimination of dysfunctional mitochondria by mitophagy (31). Confocal microscopy revealed changes in the organization of the mitochondrial network architecture in LA-Mϕ relative to Mϕ, with higher values for the morphological form factor (data not shown). However, the mitochondria in Mϕ were almost dot-like (form factor value close to 1). As such, mitochondrial budding during fission would result in higher form factor values that could be misinterpreted as a filamentous network. Transmission electron microscopy (TEM) revealed that mean mitochondrial size was smaller in LA-Mϕ than in Mϕ, with a shift in the entire distribution towards smaller sizes (data not shown). These modifications of the mitochondrial network, at the micro- and nanoscale, are entirely consistent with a more fragmentated mitochondrial network and mitophagy (27). Thus, prolonged lactic acidosis induces the depolarization of mitochondria in macrophages, resulting in a fragmented mitochondrial network.

### The Autophagic Flux in Macrophages is Enhanced During Lactic Acidosis.

Mitophagy is the selective removal of dysfunctional mitochondria by the autophagic machinery. More precisely, dysfunctional mitochondria are engulfed in autophagosomes, which then fuse with lysosomes, leading to the degradation of the content of the resulting phagolysosomes. We hypothesized that autophagic activity increases in macrophages during lactic acidosis. The number of autophagosomes in the cytoplasm of Mϕ and LA-Mϕ was determined by TEM (32). On day 3, LA-Mϕ contained larger numbers of autophagosomes than Mϕ (data not shown).

To confirm the enhanced autophagic activity in LA-Mϕ, the levels of the autophagosome-associated protein LC3-II were analyzed in the presence of bafilomycin A1 (BafA1), an inhibitor of the vacuolar H+ ATPase that prevents lysosomal acidification and interferes with autophagosome/LC3-II degradation (33). We found that LC3-II/LC3-I and LC3II/β-actin ratios were significantly higher in LA-Mϕ than in Mϕ at day 2 and 3, confirming the presence of a large number of mature autophagosomes in LA-Mϕ. The expression of p62, which binds directly to LC3 and is degraded by autophagy, also decreased strongly in LA-Mϕ, and this reduction was counterbalanced to some extent by BafA1, demonstrating that the autophagic flux was still functional in LA-Mϕ(data not shown). In addition, consistent with enhanced autophagic activity, levels of the mRNAs encoding ATG5, p62, LC3B, HMOX1 and BNIP3L1, all involved in autophagy (34), were transiently upregulated in LA-Mϕ relative to Mϕ. These observations ruled out the possibility of the increase in autophagosome numbers in LA-Mϕ resulting from a defective clearance. As previously reported (35), monocytes differentiated in the presence of GM-CSF displayed a discrete increase in ATG5 and p62 mRNA levels (data not shown). In conclusion, autophagic flux is functional and exacerbated in Mϕ under lactic acidosis.

### Macrophages Rely on Autophagy to Survive During Lactic Acidosis.

We therefore hypothesized that monocytes/macrophages use autophagy to survive prolonged exposure to lactic acidosis. To test this hypothesis, we used salinomycin and BafA1, two autophagy inhibitors that are active under acidic conditions (36-38). The addition of salinomycin or BafA1 to day 3 LA-Mϕ significantly decreased their survival (data not shown), demonstrating that Mϕ rely on autophagy to survive lactic acidosis. Autophagy therefore appears to be an adaptive protective response enabling Mϕ to survive in a lactic acidosis environment.

### Macrophages Display Metabolic and Cellular Changes Typical of Starving Cells During Lactic Acidosis.

Self-catabolic autophagy is strongly induced in response to nutrient starvation, to provide energy substrates; cellular material is, therefore sacrificed, to allow ATP production (39,40). In lactic acidosis, monocytes/Mϕ are dependent on autophagic processes to survive. We therefore hypothesized that they would have characteristics typical of cells able to cope with “starvation”, despite the presence of oxygen and nutrients.

We tested this hypothesis by investigating the impact of lactic acidosis on AMP-activated protein kinase (AMPK) phosphorylation. Nutrient deficiency depletes cellular ATP supplies, thereby activating AMPK and promoting autophagy (41,42). LA-Mϕ displayed an early increase in AMPK Thr172 phosphorylation. Lactic acidosis also upregulated AMPK expression in Mϕ (data not shown).

To confirm this observation, we analyzed the impact of lactic acidosis on intracellular levels of acetyl-coenzyme A (AcCoA), a major integrator of nutritional status at the crossroads of fat, carbohydrate, and protein catabolism (43). The decrease in intracellular AcAc levels induced by nutrient deficiency triggers autophagy (43,44). AcCoA levels were significantly lower in LA-Mϕ than in Mϕ (data not shown), demonstrating that myeloid cells subjected to lactic acidosis have characteristics similar to those of starving cells.

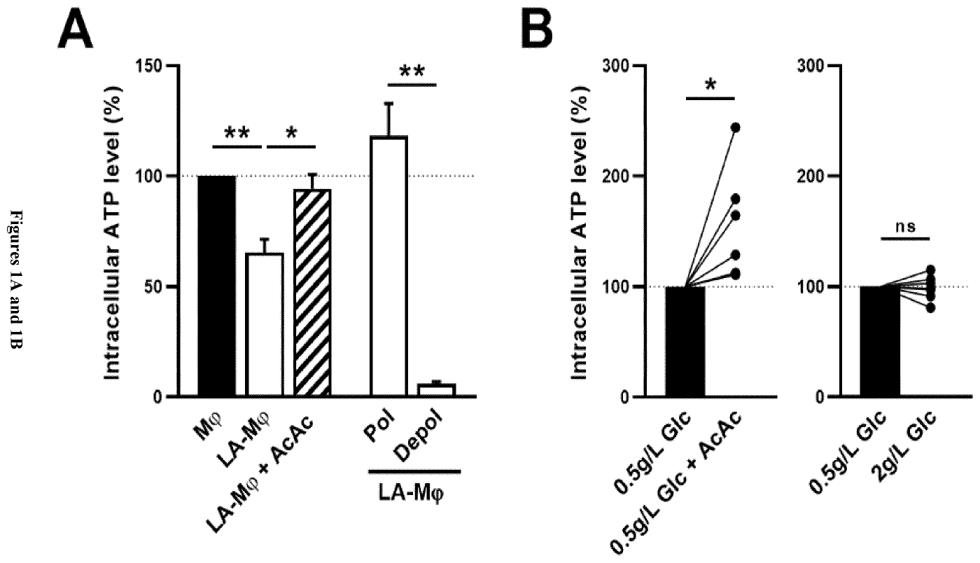

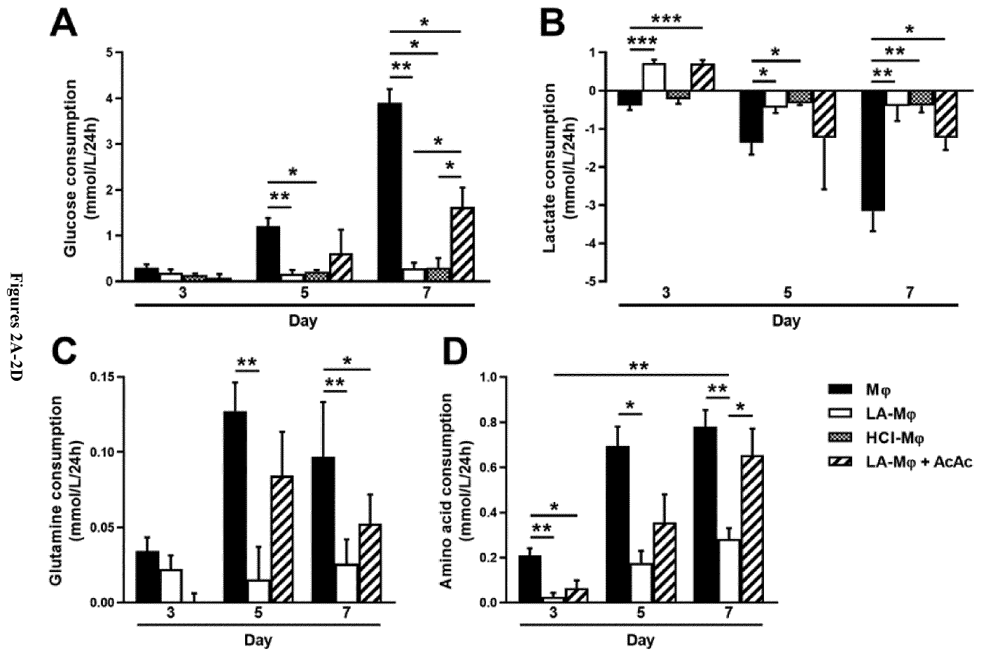

In parallel, we observed that lactic acidosis decreased the intracellular ATP content, which one was related to the FACS-sorted cell population with depolarized mitochondria (FIG. 1A), indicating that the decrease in mitochondrial membrane potential impairs oxidative ATP production leading to an energetic stress.

### Lactic Acidosis Decreases Nutrient Uptake.

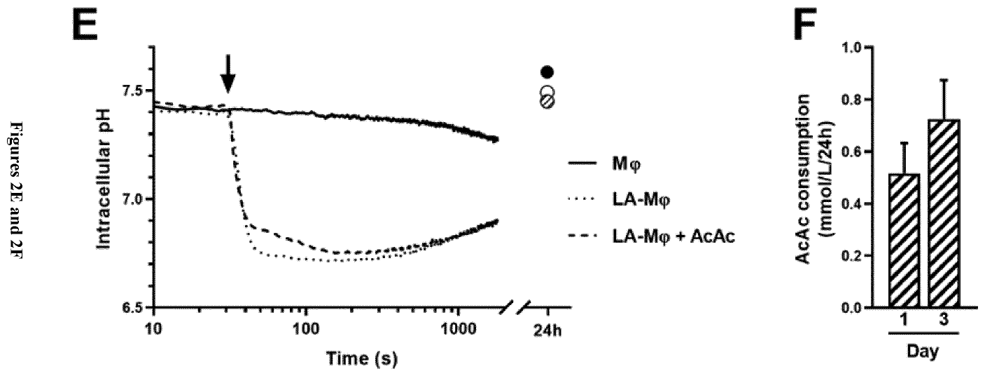

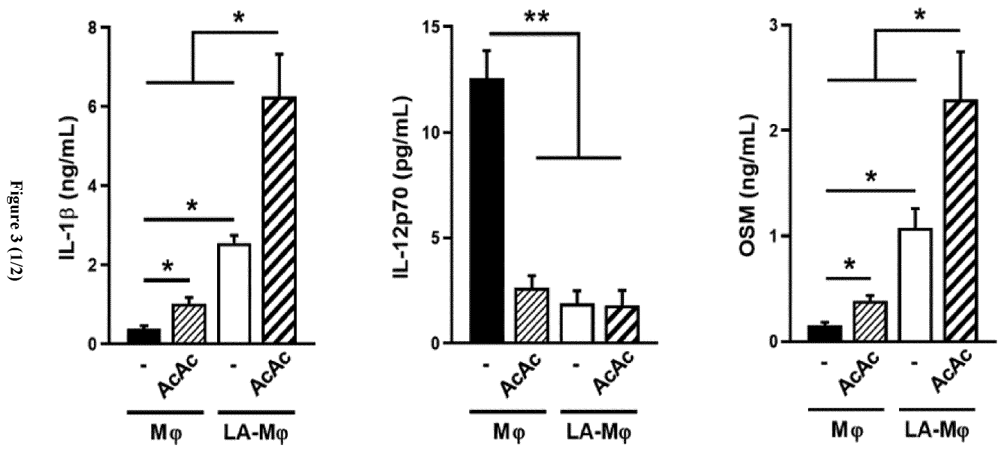

We investigated the mechanism underlying pseudostarvation in LA-Mϕ, by comparing the ability of monocytes/macrophages to consume nutrients in the presence and absence of LA. Glucose, lactate and amino acids were quantified in cell culture supernatants during the differentiation process. Under acidosis, monocytes treated with LA or HCl had low glycolysis rates. They lost their capacity to use glucose early in differentiation, and this defect was maintained until day 7 (FIG. 2A), despite cell-surface expression of the glucose transporters GLUT1, GLUT3 and GLUT5 (data not shown). Accordingly, LA-Mϕ and HCl-Mϕ released only small amounts of lactate. Following cell exposure to LA, extracellular lactate concentrations reflected the balance between LA absorption and production, and a trend towards lactate consumption was observed on day 3 in LA-Mϕ (FIG. 2B). Results also showed a decrease in the uptake of glutamine and other amino acids by LA-Mϕ compared to Mϕ (FIG. 2C-D).

In conclusion, these observations indicate that, in an acidic environment, monocytes/macrophages reduce their nutrient consumption, entering a state of pseudostarvation in which they rely on self-catabolism to survive.

### AcAc Prevents Lactic Acidosis-Induced Pseudostarvation and Mitophagy.

In fasting periods, in which carbohydrate availability is reduced, the ketone bodies AcAc and β-OHB are de novo-synthesized within hepatocyte mitochondria, via fatty acid β oxidation. They serve as vital alternative metabolic fuel for extrahepatic cells (21). They are catabolized in mitochondria, converted into AcCoA through reactions that do not require ATP (21). We investigated the possible use of ketone bodies by monocytes/macrophages, as an alternative fuel to bypass lactic acidosis-induced pseudostarvation.

Monocytes were differentiated into macrophages under lactic acidosis conditions, in the presence or absence of 5 mM AcAc (LA-Mϕ+AcAc). Surprisingly, despite the presence of LA, LA-Mϕ+AcAc did not exhibit the mitochondrial depolarization observed in the presence of LA alone (data not shown) and AcAc also restored the intracellular levels of ATP in LA-Mϕ (FIG. 1A). In parallel, AcAc protected the mitochondrial network architecture, as shown by the similarity of mitochondrial morphology and size between these cells and Mϕ (data not shown). Moreover, LA-Mϕ+AcAc displayed no reduction of the mitochondrial mass, which instead even slightly increased by day 2, accompanied by an expression of NRF2 that tend to increase relative to LA-Mϕ(data not shown). In addition, OCR consumption was also induced in LA-Mϕ+AcAc compared to LA-Mϕ. Autophagy assessed by TEM or molecularly was not upregulated in day 3 LA-Mϕ+AcAc relative to Mϕ (data not shown). LA-Mϕ may no longer need to use autophagy in the presence of AcAc, as they not only consume AcAc (FIG. 2F), but also retain a partial capacity to take up glucose and amino acids and to produce lactate (FIG. 2A-D). As expected (45), AcAc metabolism was necessary to prevent acidosis-induced mitophagy as two inhibitors of the mitochondrial thiolase ACAT1, involved in AcAc oxidation to acetylCoA, prevented the protective effect of AcAc on LA-induced mitochondrial depolarization (data not shown). Accordingly, AcAc prevented the reduction of intracellular AcCoA levels induced by LA. Finally, the immediate but short-term decrease in pHi induced by extracellular acidosis remained detectable in the presence of AcAc (FIG. 2E).

Thus, by maintaining mitochondrial integrity and cellular metabolism during lactic acidosis, AcAc appears to act as an alternative fuel, preventing cellular dysfunction due to acidic stress and increasing tissue resistance to acidosis.

### Acetoacetate Upregulates Cytokine Secretion by LA-Mϕ.

We have previously reported that human LA-Mϕ exhibit an inflammatory (TNFαhigh IL-1βhigh IL-6high) phenotype associated with a huge production of trophic factors (OSMhigh VEGFhigh) (FIG. 2) (15,16). Consistent with this observation, no endoplasmic reticulum stress was detected in LA-Mϕ by TEM (data not shown). Of note, the cytokine profiles were analyzed in response to LPS stimulation as human macrophages require stimulation to reveal their phenotype (46). We then analyzed the impact of AcAc on the phenotype of human LA-Mϕ.

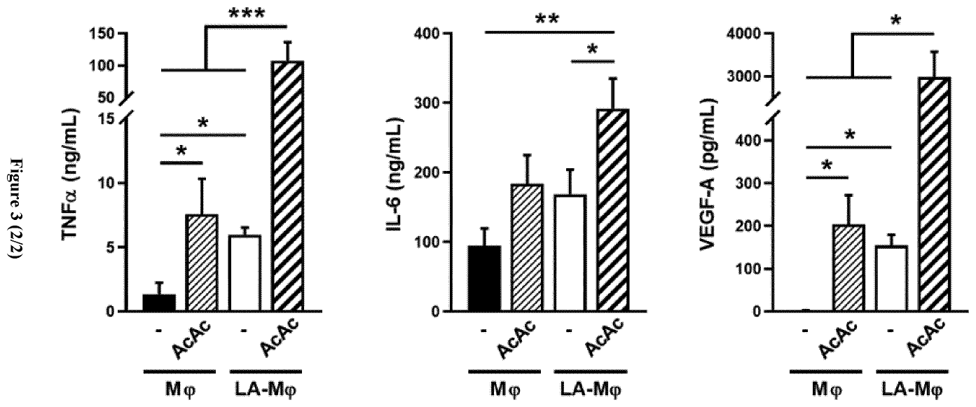

We observed that monocytes differentiated in the presence of AcAc also exhibit a TNFαhigh IL-1βhigh IL-6high OSMhigh and VEGFhigh phenotype (FIG. 3). Supporting these similar signatures, both AcAc and LA mitochondrial fuels for myeloid cells (15,45). Moreover, AcAc has been reported to enhance IL-6 and TNF production by U937 cell line (47,48) and to modulate murine macrophage properties (49). Finally, we observed that AcAc and AL acted synergistically to induce macrophages with enhanced capacity to produce inflammatory cytokines and trophic factors. The capacity of AcAc to protect the energetic and metabolic functions of macrophages under acidosis and the similar signature printed AcAc and AL on macrophages help to explain these results (FIG. 3). In conclusion, these results show that AcAc not only bypasses LA-induced macrophage starvation and autophagy and but also improves their ability to produce cytokines.

### Acetoacetate Reduces Septic Shock-Associated Mitochondrial Depolarization and Restores Intracellular ATP Content

In sepsis, mitochondrial dysfunction (mitochondrial depolarization, reduction of mitochondrial respiration, ATP production and mitochondrial mass) occurs early, and its persistence contributes to organ failure and poor clinical prognosis (50-52). The cause of the mitochondrial dysfunction in sepsis remains undetermined and a role for inflammatory mediators has been suggested (53). Sepsis is also associated with an increase in lactate production (with low levels of clearance by gluconeogenesis), which can lead to systemic acidosis (pH<7.38) and is correlated with disease severity, morbidity and mortality. Our findings on human myeloid cells chronically exposed to LA strongly suggest that acidosis is a major contributor of the mitochondrial dysfunction observed in situations in which extracellular acidosis occurs, such as sepsis.

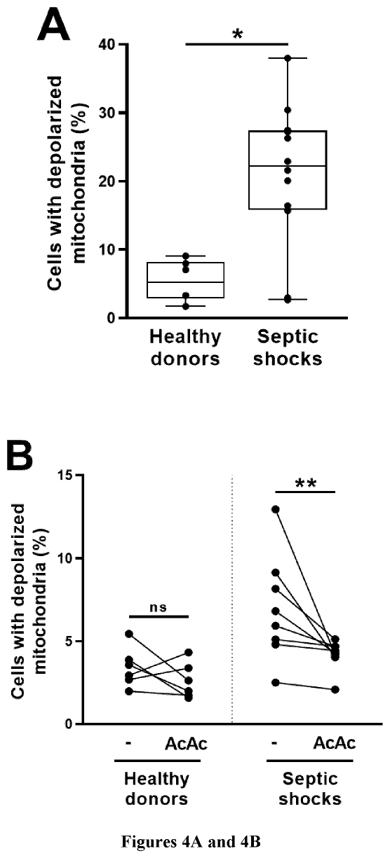

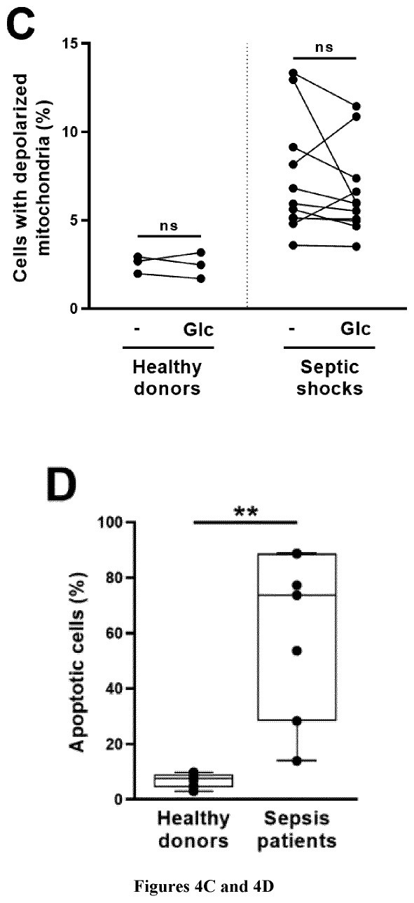

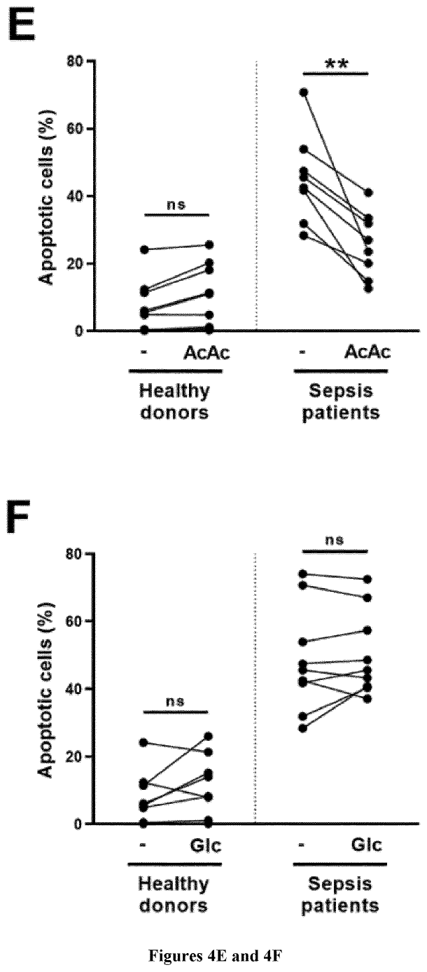

We thereby analysed whether AcAc could restore the mitochondrial function in septic shock patients. As previously reported (54), we observed that peripheral blood mononuclear cells (PBMC) from septic shock patients exhibited a significant increase in the percentage of cells exhibiting depolarized mitochondria compared to healthy subjects (FIG. 4A).

In order to determine whether AcAc may restore mitochondrial function in cells from sepsis patients, PBMC from healthy subjects and sepsis patients were exposed for 24 h in a medium supplemented with 5 mM AcAc (AcAc) or enriched in glucose (4 g/L). We report that AcAc (FIG. 4B), but not a high glucose containing medium (FIG. 4C), significantly reduced the percentage of cells with depolarized mitochondria in sepsis patients.

Finally, we observed that AcAc, but not glucose, increased the levels of intracellular ATP in PBMC isolated from septic shock patients (FIG. 1B).

AcAc Protects PBMC from Septic Shock Patients from Death by Apoptosis.

Because of mitochondrial dysfunctions, PBMC from septic shock patients die spontaneously by apoptosis (FIG. 4D). Because AcAc fuels and restores mitochondrial metabolism in patient “PBMCs (see above), we evaluated whether it could also protect them from apoptosis. We observed that the presence of AcAc, but not Glc, drastically protects patient PBMCs from death (FIGS. 4E and F), showing that AcAc could be used as a fuel, rather than Glc, to rescue cells from patients in septic shock.

### Discussions and Conclusions

Lactic acidosis is a characteristic of injured tissues, such as areas of wound repair and solid tumor microenvironments (6,17). The metabolic strategies enabling tumor cells to survive in acidic environments have been widely studied (4,17-20), but little is known about the ability of human monocytes/ϕ to cope with these hostile conditions. We show here that a prolonged exposure of Mϕ to lactic acidosis leads to mitochondrial depolarization and a large decrease in nutrient consumption, highlighting the essential role of autophagy in cell survival. We also report that the ketone body acetoacetate can fuel myeloid cells during acidosis, maintaining cellular energy metabolism and mitochondrial integrity and function. Our results demonstrate that (i) prolonged lactic acidosis alters mitochondrial functions and metabolism of Mϕ and that (ii) acetoacetate, by providing cells with an alternative fuel, maintains mitochondrial integrity, thus improving cell and tissue tolerance to acidic stress.

In conditions of extracellular acidosis, monocytes/Mϕ enter a pseudostarvation state, despite the presence of nutrients and oxygen. Tumor cells have also been shown to respond to acidosis by entering a pseudostarvation phase (18,41) associated with a large decrease in glucose uptake, glycolysis, and amino-acid consumption (18,20,52). As in myeloid cells, extracellular acidification also resulted in a steep drop in pHi in tumor cells (18,55-57). It has been suggested that this decrease in pHi inhibits the activity of enzymes involved in glycolysis (58) and acidosis-sensitive glutamine pumps (59,60).

Sustained autophagy seems essential for tumor cell survival in an acidic microenvironment (4). Melanoma cells subjected to acidic stress display a pseudostarvation response and rely on autophagy to survive (18,36). We showed that non-proliferating human myeloid cells also rely on autophagy to survive in an acidic environment. This autophagic phase was transient and Mϕ gradually recovered their capacity to take up amino acids.

AcAc and β-OHB are the main ketone bodies present in the body. They serve as alternative fuel for the mitochondria of extrahepatic cells in cases of starvation or carbohydrate restriction2l. The oxidation of AcAc into AcCoA is mediated by a CoA transferase (SCOT) that generates AcAc-CoA, followed by thiolases, yielding two molecules of AcCoA, which enter the TCA cycle (21). AcAc oxidation does not require ATP. AcAc oxidative flux occurs due to mass action: an abundant supply of AcAc and the rapid consumption of AcCoA via citrate synthase in the TCA cycle favors AcAc-CoA formation by OXCT1 (21). By contrast, hexokinase and acyl-CoA synthetases require ATP to generate and process AcCoA from glucose and fatty acids, respectively. This may explain why cells continue to be able to consume AcAc during lactic acidosis, whereas they are unable to metabolize glucose and amino acids.

The balance between energy demand and nutrient supply is regulated by mitochondrial dynamics (61). Nutrient starvation generally leads to an inhibition of mitochondrial fission and mitochondrial elongation, due to unopposed fusion. This elongation prevents the removal of mitochondria by mitophagy and increases the capacity for ATP synthesis, thereby satisfying the demand for ATP during periods of limited nutrient availability (61). By contrast, despite displaying the biochemical and morphological characteristics of starving cells, Mϕ subjected to acidosis have depolarized mitochondria, with higher rates of fission events (as shown by the decrease in mitochondrial size and the presence of a fragmented mitochondrial network). It is tempting to speculate that, under lactic acidosis, the increase in cytosolic proton concentration leading to the decrease of pHi also affects mitochondrial membrane potential. Similarly, a reduction of the pHi has been reported to trigger autophagy and mitophagy (18,55-57). This decrease is transient, with the pHi returning to values similar to those in cells maintained in a standard medium after 24 hours, suggesting that the pumps and proteins controlling pHi are very effective in human myeloid cells. In addition to having a direct effect on mitochondrial membrane potential, this decrease in pHi also inhibits glycolysis and amino-acid catabolism. This inhibition of the catabolic pathways responsible for supplying the TCA cycle and then the mitochondrial complexes with reducing equivalents (NADH, H+, FADH2) may decrease the ability of the respiratory chain to maintain the mitochondrial membrane potential.

Importantly, even though AcAc prevents acidosis-induced pseudostarvation, mitochondrial depolarization and autophagy, it does not prevent the drop of the pHi induced by lactic acidosis. We therefore suggest that AcAc, by feeding the TCA cycle and boosting mitochondrial respiration and biogenesis, prevents the depolarization of the mitochondrial membrane induced by the fall in pHi. In conclusion, AcAc protects cells from the metabolic stress induced by lactic acidosis (a) by providing them with fuel for the maintenance of their energy metabolism and (b) by ensuring mitochondrial integrity and function.

Many clinical situations in medicine are associated with tissue acidosis (areas of wound repair, solid tumor microenvironments) or systemic acidosis (septic shock). It is tempting to speculate that ketogenesis could protect Mϕ from acidic stress and stimulate their functions. For instance, in sepsis, mitochondrial dysfunction (mitochondrial depolarization, reduction of mitochondrial respiration and ATP production) occurs early, and its persistence contributes to organ failure and poor clinical prognosis (50-52). The cause of the mitochondrial dysfunction in sepsis remains undetermined, but a role for inflammatory mediators has been suggested (53). Sepsis is also associated with an increase in lactate production (with low levels of clearance by gluconeogenesis), which can lead to systemic acidosis (pH<7.38) and is correlated with disease severity, morbidity, and mortality. Our findings on human myeloid cells chronically exposed to LA strongly suggest that acidosis is a major contributor of the mitochondrial dysfunction observed in situations in which extracellular acidosis occurs, such as sepsis.

Signs of cell starvation are observed during sepsis, together with a reduction of glucose, fatty acid, and amino acid catabolism, and hyperglycemia (53). The observation that myeloid cells fail to take up nutrients during acidosis may, therefore, help to explain some of the metabolic changes associated with sepsis. Indeed, supplementation with glucose or nutrients is harmful in mouse models of bacterial sepsis, as such supplementation decreases host tolerance and increases host mortality (62).

None of the treatments proposed for protecting mitochondria during sepsis are really efficient (63). However, fasting and ketogenesis appear to be essential to animal survival and increase the ability of tissues to tolerate damage due to inflammation (62). We show that AcAc protects mitochondria by enabling them to remain metabolically active in an acidic environment. As ketogenesis is affected in sepsis patients (22), AcAc appears as a candidate of choice for increasing cell tolerance to acidic stress, thereby preventing organ dysfunction and failure.

In conclusion, we report that non-proliferating human monocytes/Mb cope with an acidic microenvironment through a transient phase of autophagy and mitophagy, explaining their survival and ability to perform their functions in injured tissues and tumor lesions. We identify AcAc as a unique molecule that supports cellular metabolism during extracellular lactic acidosis, thereby maintaining mitochondrial integrity and preventing the need for a transient, but deleterious autophagic process. These results highlight the potential role of AcAc as a natural metabolite capable of protecting cells and enhancing host tolerance in pathological situations associated with acute or chronic acidosis like sepsis.

### REFERENCES

Throughout this application, various references describe the state of the art to which this invention pertains. The disclosures of these references are hereby incorporated by reference into the present disclosure.
1. Dubos, R. THE MICRO-ENVIRONMENT OF INFLAMMATION or METCHNIKOFF REVISITED. The Lancet 266, xxiv-5 (1955).
2. Simmen, H.-P. & Blaser, J. Analysis of pH and p02 in abscesses, peritoneal fluid, and drainage fluid in the presence or absence of bacterial infection during and after abdominal surgery. The American Journal of Surgery 166, 24-27 (1993).
3. Gallagher, F. A. et al. Magnetic resonance imaging of pH in vivo using hyperpolarized 13C-labelled bicarbonate. Nature 453, 940-943 (2008).
4. Corbet, C. & Feron, O. Tumour acidosis: from the passenger to the driver's seat. Nature Reviews Cancer 17, 577-593 (2017).
5. Vander Heiden, M. G., Cantley, L. C. & Thompson, C. B. Understanding the Warburg effect: the metabolic requirements of cell proliferation. Science 324, 1029-1033 (2009).
6. Sun, S., Li, H., Chen, J. & Qian, Q. Lactic Acid: No Longer an Inert and End-Product of Glycolysis. Physiology (Bethesda) 32, 453-463 (2017).
7. Casey, J. R., Grinstein, S. & Orlowski, J. Sensors and regulators of intracellular pH. Nat. Rev. Mol. Cell Biol. 11, 50-61 (2010).
8. Gottfried, E. et al. Tumor-derived lactic acid modulates dendritic cell activation and antigen expression. Blood 107, 2013-2021 (2006).
9. Erra Diaz, F., Dantas, E. & Geffner, J. Unravelling the Interplay between Extracellular Acidosis and Immune Cells. Mediators of Inflammation 2018, 1-11 (2018).
10. Okabe, Y. & Medzhitov, R. Tissue biology perspective on macrophages. Nat. Immunol. 17, 9-17 (2016).
11. Sica, A. & Mantovani, A. Macrophage plasticity and polarization: in vivo veritas. J. Clin. Invest. 122, 787-795 (2012).
12. Xue, J. et al. Transcriptome-based network analysis reveals a spectrum model of human macrophage activation. Immunity 40, 274-288 (2014).
13. Shaw, T. N. et al. Tissue-resident macrophages in the intestine are long lived and defined by Tim-4 and CD4 expression. J. Exp. Med. 215, 1507-1518 (2018).
14. Mantovani, A., Marchesi, F., Malesci, A., Laghi, L. & Allavena, P. Tumour-associated macrophages as treatment targets in oncology. Nat Rev Clin Oncol 14, 399-416 (2017).
15. Colegio, O. R. et al. Functional polarization of tumour-associated macrophages by tumour-derived lactic acid. Nature 513, 559-563 (2014).
16. Paolini, L. et al. Lactic Acidosis Together with GM-CSF and M-CSF Induces Human Macrophages toward an Inflammatory Protumor Phenotype. Cancer Immunology Research 8, 383-395 (2020).
17. Baldini, N. et al. Metabolism and microenvironment in cancer plasticity. Cancer & Metabolism 4, 1 (2016).
18. Marino, M. L. et al. Autophagy is a protective mechanism for human melanoma cells under acidic stress. J. Biol. Chem. 287, 30664-30676 (2012).
19. Xie, J. et al. Beyond Warburg effect—dual metabolic nature of cancer cells. Sci Rep 4, 4927 (2014).
20. Chen, J. L.-Y. et al. The genomic analysis of lactic acidosis and acidosis response in human cancers. PLoS Genet. 4, e1000293 (2008).
21. Puchalska, P. & Crawford, P. A. Multi-dimensional Roles of Ketone Bodies in Fuel Metabolism, Signaling, and Therapeutics. Cell Metab. 25, 262-284 (2017).
22. Ohtoshi, M. et al. Ketogenesis during sepsis in relation to hepatic energy metabolism. Res Exp Med (Berl) 184, 209-219 (1984).
23. Desquiret-Dumas, V. et al. Resveratrol induces a mitochondrial complex I-dependent increase in NADH oxidation responsible for sirtuin activation in liver cells. J. Biol. Chem. 288, 36662-36675 (2013).
24. Iannetti, E. F., Smeitink, J. A. M., Beyrath, J., Willems, P. H. G. M. & Koopman, W. J. H. Multiplexed high-content analysis of mitochondrial morphofunction using live-cell microscopy. Nat Protoc 11, 1693-1710 (2016).
25. Springer, M. Z. & Macleod, K. F. In Brief: Mitophagy: mechanisms and role in human disease. J. Pathol. 240, 253-255 (2016).
26. Gottlieb, R. A. & Bernstein, D. Mitochondrial remodeling: Rearranging, recycling, and reprogramming. Cell Calcium 60, 88-101 (2016).
27. Twig, G. & Shirihai, O. S. The interplay between mitochondrial dynamics and mitophagy. Antioxid. Redox Signal. 14, 1939-1951 (2011).
28. Xiao, B., Deng, X., Zhou, W. & Tan, E.-K. Flow Cytometry-Based Assessment of Mitophagy Using MitoTracker. Front Cell Neurosci 10, 76 (2016).
29. Lee, M. H. et al. Mitochondria-immobilized pH-sensitive off-on fluorescent probe. J. Am. Chem. Soc. 136, 14136-14142 (2014).
30. Tal, M. C. et al. Absence of autophagy results in reactive oxygen species-dependent amplification of RLR signaling. Proc. Natl. Acad. Sci. U.S.A. 106, 2770-2775 (2009).
31. Yu, T., Wang, L. & Yoon, Y. Morphological control of mitochondrial bioenergetics. Front Biosci (Landmark Ed) 20, 229-246 (2015).
32. Xie, Z., Nair, U. & Klionsky, D. J. Dissecting autophagosome formation: the missing pieces. Autophagy 4, 920-922 (2008).
33. Mizushima, N., Yoshimori, T. & Levine, B. Methods in mammalian autophagy research. Cell 140, 313-326 (2010).
34. Dikic, I. & Elazar, Z. Mechanism and medical implications of mammalian autophagy. Nat. Rev. Mol. Cell Biol. 19, 349-364 (2018).
35. Zhang, Y., Morgan, M. J., Chen, K., Choksi, S. & Liu, Z. Induction of autophagy is essential for monocyte-macrophage differentiation. Blood 119, 2895-2905 (2012).
36. Pellegrini, P. et al. Tumor acidosis enhances cytotoxic effects and autophagy inhibition by salinomycin on cancer cell lines and cancer stem cells. Oncotarget 7, 35703-35723 (2016).
37. Allen, G. F. G., Toth, R., James, J. & Ganley, I. G. Loss of iron triggers PINKI/Parkin-independent mitophagy. EMBO Rep. 14, 1127-1135 (2013).
38. Benischke, A.-S. et al. Activation of mitophagy leads to decline in Mfn2 and loss of mitochondrial mass in Fuchs endothelial corneal dystrophy. Sci Rep 7, 6656 (2017).
39. Palikaras, K., Lionaki, E. & Tavernarakis, N. Balancing mitochondrial biogenesis and mitophagy to maintain energy metabolism homeostasis. Cell Death Differ. 22, 1399-1401 (2015).
40. Kaur, J. & Debnath, J. Autophagy at the crossroads of catabolism and anabolism. Nat. Rev. Mol. Cell Biol. 16, 461-472 (2015).
41. Chen, J. L.-Y. et al. Lactic acidosis triggers starvation response with paradoxical induction of TXNIP through MondoA. PLoS Genet. 6, e1001093 (2010).
42. Garcia, D. & Shaw, R. J. AMPK: Mechanisms of Cellular Energy Sensing and Restoration of Metabolic Balance. Mol. Cell 66, 789-800 (2017).
43. Pietrocola, F., Galluzzi, L., Bravo-San Pedro, J. M., Madeo, F. & Kroemer, G. Acetyl coenzyme A: a central metabolite and second messenger. Cell Metab. 21, 805-821 (2015).
44. Mariño, G. et al. Regulation of autophagy by cytosolic acetyl-coenzyme A. Mol. Cell 53, 710-725 (2014).
45. Puchalska, P. et al. Hepatocyte-Macrophage Acetoacetate Shuttle Protects against Tissue Fibrosis. Cell Metab. 29, 383-398.e7 (2019).
46. Izquierdo, E. et al. Reshaping of Human Macrophage Polarization through Modulation of Glucose Catabolic Pathways. J. Immunol. 195, 2442-2451 (2015).
47. Jain, S. K., Kannan, K., Lim, G., McVie, R. & Bocchini, J. A. Hyperketonemia Increases Tumor Necrosis Factor-Secretion in Cultured U937 Monocytes and Type 1 Diabetic Patients and Is Apparently Mediated by Oxidative Stress and cAMP Deficiency. Diabetes 51, 2287-2293 (2002).
48. Jain, S. K. et al. Elevated Blood Interleukin-6 Levels in Hyperketonemic Type 1 Diabetic Patients and Secretion by Acetoacetate-Treated Cultured U937 Monocytes. Diabetes Care 26, 2139-2143 (2003).
49. Puchalska, P. et al. Hepatocyte-Macrophage Acetoacetate Shuttle Protects against Tissue Fibrosis. Cell Metab. 29, 383-398.e7 (2019).
50. Arulkumaran, N. et al. MITOCHONDRIAL FUNCTION IN SEPSIS. Shock 45, 271-281 (2016).
51. Singer, M. The role of mitochondrial dysfunction in sepsis-induced multi-organ failure. Virulence 5, 66-72 (2014).
52. Brealey, D. & Singer, M. Mitochondrial Dysfunction in Sepsis. Curr Infect Dis Rep 5, 365-371 (2003).
53. Van Wyngene, L., Vandewalle, J. & Libert, C. Reprogramming of basic metabolic pathways in microbial sepsis: therapeutic targets at last? EMBO Mol Med 10, (2018).
54. Weiss S L et al. Mitochondrial dysfunction in peripheral blood mononuclear cells in pediatric septic shock. Pediatr Crit Care Med. 16, e4-e12 (2015).
55. Berezhnov, A. V. et al. Intracellular pH Modulates Autophagy and Mitophagy. J. Biol. Chem. 291, 8701-8708 (2016).
56. Rotin, D., Robinson, B. & Tannock, I. F. Influence of hypoxia and an acidic environment on the metabolism and viability of cultured cells: potential implications for cell death in tumors. Cancer Res. 46, 2821-2826 (1986).
57. Wahl, M. L., Pooler, P. M., Briand, P., Leeper, D. B. & Owen, C. S. Intracellular pH regulation in a nonmalignant and a derived malignant human breast cell line. J. Cell. Physiol. 183, 373-380 (2000).
58. Xie, J. et al. Beyond Warburg effect—dual metabolic nature of cancer cells. Sci Rep 4, 4927 (2014).
59. Evans, K. et al. Acidosis-sensing glutamine pump SNAT2 determines amino acid levels and mammalian target of rapamycin signalling to protein synthesis in L6 muscle cells. J. Am. Soc. Nephrol. 18, 1426-1436 (2007).
60. Evans, K. et al. Inhibition of SNAT2 by metabolic acidosis enhances proteolysis in skeletal muscle. J. Am. Soc. Nephrol. 19, 2119-2129 (2008).
61. Liesa, M. & Shirihai, O. S. Mitochondrial dynamics in the regulation of nutrient utilization and energy expenditure. Cell Metab. 17, 491-506 (2013).
62. Wang, A. et al. Opposing Effects of Fasting Metabolism on Tissue Tolerance in Bacterial and Viral Inflammation. Cell 166, 1512-1525.e12 (2016).
63. Leite, H. P. & de Lima, L. F. P. Metabolic resuscitation in sepsis: a necessary step beyond the hemodynamic? J Thorac Dis 8, E552-557 (2016).

Shankar-Har et al. Developing a New Definition and Assessing New Clinical Criteria for Septic Shock: For the Third International Consensus Definitions for Sepsis and Septic Shock (Sepsis-3). JAMA 2016.

1. (canceled)

2. (canceled)

3. (canceled)

4. (canceled)

5. (canceled)

6. A method for treating an acidosis related disease in a subject in need thereof comprising administering to said subject in need thereof a therapeutically effective amount of acetoacetate (AcAc).

7. A therapeutic composition comprising acetoacetate (AcAc),
wherein the therapeutic composition is formulated for administration to a subject suffering from acidosis.

8. The method according to the claim 6, wherein the acidosis related disease is a lactic acidosis related disease.

9. The method according to the claim 6 wherein the acidosis related disease is tissular hypoxia, CO intoxication, a hemopathy, a cancer, drug intoxication, acute liver failure or pheochromocytoma.

10. The method according to claim 9 wherein the tissular hypoxia is sepsis or septic shock.

11. The method of claim 9, wherein the cancer is leukemia, lymphoma or metastatic cancer.

12. The method of claim 9, wherein the drug intoxication is caused by metformin.

13. The according to claim 6, wherein the acetoacetate (AcAc) is ethyl acetoacetate or sodium acetoacetate.
